(19) United States
(12) Patent Application Publication
(10) Pub. No.: US 2015/0297093 A1
(43) Pub. Date: Oct. 22, 2015

(54) Flow rate sensor system and method for non-invasively measuring the flow rate of a bodily fluid

(71) Applicant: Vivonics, Inc., Waltham, MA (US)

(72) Inventors: James H. Goldie, Lexington, MA (US); Thieu Q. Truong, North Easton, MA (US); Minh Duong, Worcester, MA (US); Thomas Russell, New Providence, NJ (US)

(73) Assignee: Vivonics, Inc., Waltham, MA (US)

(21) Appl. No.: 14/120,048

(22) Filed: Apr. 18, 2014

(51) Int. Cl. A61B 5/01; A61B 5/00; A61B 5/026

(52) U.S. Cl. CPC A61B 5/01; A61B 5/026; A61B 5/7278; A61B 5/0008

(57) ABSTRACT

A flow rate sensor system for non-invasively measuring the flow rate of a bodily fluid. The system includes an encapsulated implant having a flow tube having an inlet and an outlet configured to receive a flow of a bodily fluid. A heating element externally coupled to the flow tube is configured to dissipate heat at a predetermined rate over a predetermined amount of time. A temperature sensor externally coupled to the heating element is configured to measure a temperature rise of the heating element over the predetermined amount of time. An implant microcontroller coupled to the temperature sensor is configured to determine the flow rate of the bodily fluid in the flow tube from the measured temperature rise and a curve fit to a stored set of previously obtained calibration measurements. An implant power and communication subsystem is coupled to the implant microcontroller configured to wirelessly receive power and wirelessly transmit and receive data. The system also includes an external device having an external microcontroller and an external power and communication subsystem coupled to the external microcontroller configured to wirelessly deliver power to the implant power and communication subsystem and transmit and receive data to and from the implant power and communication subsystem.

### GOVERNMENT RIGHTS

This invention was made with U.S. Government support under Grant No. 6R44NS056628-04 awarded by the National Institutes of Health. The Government may have certain rights in certain aspects of the subject invention.

### FIELD OF THE INVENTION

This invention relates to a flow rate sensor system and method for measuring the flow rate of a bodily fluid.

### COMPUTER PROGRAM LISTING APPENDIX

A computer program listing appendix is filed herewith on compact disk. The material on the compact disk is hereby incorporated by reference. Two identical compact disks have been submitted. Each compact disk contains two files entitled source implant.txt and source external.txt. The two disks were created on Mar. 21, 2014.

### BACKGROUND OF THE INVENTION

Drainage of cerebrospinal fluid (CSF) is one major life-sustaining therapy which may be used for patients with congenital or acquired hydrocephalus or patients with serious head injuries. Ventriculo-peritoneal (VP) shunt placement for CSF drainage is a common procedure in neurosurgery. However, shunt failure is common and shunt revision surgery is even more common than initial placement. One study of shunt-related deaths from January 1990 to July 1996 found that children are dying of shunt failure and that early detection could prevent many of these deaths. Another study of all shunt procedures performed between January 1996 and December 2005, excluding temporary shunts such as external ventricular drains (EVDs), found that the median shunt survival life span was 398 days. These results are in good agreement yet another study that suggests failure rates of 25 to 40 percent in the first year and failure of seventy percent of shunts by five years. These findings point to the importance of the need for a non-invasive and convenient system and method for monitoring shunt function.

Invasive surgery allows direct observation of the shunt and its flow behavior when it is allowed to drain into a collection vessel. This is different than measuring the flow rate when the shunt is draining into the peritoneum, but it does allow the surgeon to check patency of the shunt and provide an indication of current flow.

The ShuntCheck by NeuroDX Development (Bensalem, Pa. 19020) has been shown in clinical studies to provide a means for assessing CSF flow through a VP Shunt. The ShuntCheck uses thermal techniques for determining that CSF flow is present in the shunt. The ShuntCheck relies on a disposable temperature sensor placed over the skin proximate the shunt tubing. An ice cube is placed over the shunt and the effect on the temperature of the skin close to the shunt downstream of the ice cube is monitored. The ShuntCheck has the advantage that it is not implanted, but it has the disadvantage that it is unable to provide a quantitative measure of flow rate.

Another conventional quantitative flow measuring device for measuring the flow of CSF in VP shunt tubing uses an implantable device that produces a bubble in the shunt tubing by electrolysis. The bubble is then detected by an electrode arrangement using electric impedance or ultrasonically with a Doppler probe. Extracorporeal high-frequency transmission supplies the energy for electrolysis and flow may be calculated based on the velocity of bubble flow in the tubing.

Another conventional VP shunt pressure sensor determines pressure from deflection of a capacitive membrane. The shunt flow sensor membrane has a vacuum on the side of the membrane that is not in contact with fluid. Thus, the device is measuring pressure relative to vacuum.

Yet another conventional implanted intracranial pressure (ICP) sensor relies on the deflection of a membrane and the resultant change in the resonance of an LC-circuit.

However, none of the devices discussed above have yet to be demonstrated in humans to provide sufficiently reliable and accurate quantitative information on shunt function to be adopted for clinical use.

Current techniques to evaluate implanted shunts may include cranial imaging techniques, such as ultrasonography, computer assisted tomography (CAT), magnetic resonance imaging (MRI), and the like. Such techniques require relatively expensive equipment typically only available in hospitals. Intracranial pressure monitoring is currently available through implanted catheters and transducers, typically in an intensive care unit. Thus, clinicians rely on reports of symptoms from the patient, imaging of the ventricles, or the use of invasive devices to treat diseases related to CSF flow through VP shunts. Additionally, the progression of disease and injuries cannot be studied extensively because of the lack of shunt flow data.

Therefore, there is a need for an implanted sensor system capable of determining and reporting flow rate of a bodily fluid, such as CSF, that can be queried transcutaneously to allow the clinician to noninvasively assess shunt function.

### SUMMARY OF THE INVENTION

This invention features a flow rate sensor system for non-invasively measuring the flow rate of a bodily fluid. The system includes an encapsulated implant having a flow tube having an inlet and an outlet configured to receive a flow of a bodily fluid. A heating element externally coupled to the flow tube is configured to dissipate heat at a predetermined rate over a predetermined amount of time. A temperature sensor externally coupled to the heating element is configured to measure a temperature rise of the heating element over the predetermined amount of time. An implant microcontroller coupled to the temperature sensor is configured to determine the flow rate of the bodily fluid in the flow tube from the measured temperature rise of the heating element over the predetermined amount of time and a curve fit to a stored set of previously obtained calibration measurements. An implant power and communication subsystem coupled to the implant microcontroller is configured to wirelessly receive power and wirelessly transmit and receive data.

The system also includes an external device having an external microcontroller and an external power and communication subsystem coupled to the external microcontroller configured to wirelessly deliver power to the implant power and communication subsystem and transmit and receive data to and from the implant power and communication subsystem.

In one embodiment, the temperature sensor may include a thermistor or a resistance temperature detector (RTD). The thermistor may be configured as both the temperature sensor and the heating element. The temperature sensor may include a thermocouple. The heating element may include a surface mount resistor. The heating element may include a coil of electrically conductive wire or a printed circuit heater. The heating element may be directly attached to the external surface of the flow tube. The system may include a thermal insulator configured to thermally isolate the heating element and the temperature sensor from cooling paths other than the direct cooling path to the bodily fluid in the flow tube. The thermal insulator may include an insulation layer over the heating element and the temperature sensor. The thermal insulator may include a sealed volume of air surrounding the heating element and the temperature sensor. The flow through flow tube may be comprised of a thin wall of polymer material with low thermal conductivity configured to limit heat transfer along a length and a circumference of the tube while maintaining heat transfer in a radial direction to the fluid. The bodily fluid may include one or more of: cerebrospinal fluid (CSF), bile, blood, and urine. The encapsulated implant may be coupled to a shunt, tube, vessel or catheter implanted in a human body or an animal. The shunt may include one or more of: a ventriculo-peritoneal (VP) shunt, ventroarterial shunt, and lumboperitoneal shunt. The encapsulated implant may be coupled to a distal catheter of the shunt. The encapsulated implant may be coupled to a proximal catheter of the shunt. The heating element and the temperature sensor may be located proximate the outlet. The heating element and the temperature sensor may be located proximate the inlet. The heating element and the temperature sensor may be located between the inlet and the outlet. The external power and communication subsystem includes an external coil coupled to the external microcontroller and the implant power and communication subsystem includes an implant coil coupled to the microcontroller. The implant coil of the encapsulated implant may be located using the magnitude of the induced voltage wirelessly sent from the implant coil to the external coil. The external coil may be positioned proximate and in alignment with the implant coil to achieve sufficient inductive coupling between the external coil and the implant coil. The external coil may be remotely located from and tethered to the external power and communication subsystem. The implant coil may be integrated with the encapsulated implant. The implant coil may be remotely located from and tethered to the encapsulated implant. The external power and communication subsystem may include a resonant circuit comprised of the external coil and a capacitor, and a source of low-level voltage pulses, the external device resonant circuit configured to provide sinusoidal current in the external coil of sufficient amplitude to induce sufficient sinusoidal voltage in the implant coil. The implant power and communication subsystem may include an implant resonant circuit comprised of the implant coil and a capacitor having a resonance frequency closely matched to the resonance frequency of the external resonant circuit to maintain sufficient AC voltage amplitude to power the implant power and communication subsystem and to enable communication between the external power and communication subsystem and implant power and communication subsystem. The implant power and communication subsystem may be configured to convert induced sinusoidal voltages in the implant coil to a highly regulated DC voltage over the range of loading conditions to power the heating element, the temperature sensor, the microcontroller, and components of the implant power and communication subsystem. The external power communication subsystem may be configured to enable the external microcontroller to communicate data to the implant power and communication subsystem by changing the voltage supplied to the resonant circuit of the external power and communication subsystem to modulate the amplitude of the voltage induced in the implant coil and use that change in voltage to represent different binary states. The implant power and communication subsystem may transmit binary values serially to the external power and communication subsystem by sequentially applying and removing an electrical load from the implant coil to induce changes in voltage in the external coil that are decoded into data by the external microcontroller. The external power and communication subsystem may include a sense resistor configured to measure change in the amplitude of the current in external power and communication subsystem resulting from changes in the induced voltage in the external coil. The external microcontroller may be coupled to the series resistor and may be configured to decode changes in the current of the external power and communication subsystem into data. The implant microcontroller may be configured to store the set of previously obtained calibration measurements relating heating element temperature rise to flow rate. The implant microcontroller may be configured to determine the flow rate from the measured temperature rise when temperature of the heating element is determined to be no longer rising to minimize the length of time needed to determine the flow rate, the amount of heat generated by the heating device, and the amount of heat delivered to a patient. The implant microcontroller may be configured to store identification information associated with the encapsulated implant. The implant microcontroller may be configured to use the mean value of a set of temperature rise samples obtained over the predetermined amount of time as the temperature rises to determine the flow rate of the bodily fluid in order to increase the signal to noise ratio. The implant microcontroller may be configured to use a weighted average of a set of temperature rise samples obtained over a predetermined amount of time as the temperature rises to determine the flow rate of the bodily fluid in order to increase the signal to noise ratio. The encapsulated implant may be implanted in a human body. The external device may include a smart device including a flow sensor

App and a tethered external coil. The external device may include a display for displaying one or more of: the measured flow rate, the predetermined amount of time, induced voltage on the implant coil, and identification information associated with the encapsulated implant.

In another aspect, a flow rate sensor system for non-invasively measuring the flow rate of a bodily fluid is featured. The system includes an encapsulated implant having a flow tube having an inlet and an outlet configured to receive a flow of a bodily fluid. A heating element externally coupled to the flow tube is configured to dissipate heat at a predetermined rate over a predetermined temperature rise of the heating element. A temperature sensor externally coupled to the heating element is configured to measure a temperature drop of the heating element over a predetermined amount of time of cooling. An implant microcontroller coupled to the temperature sensor is configured to determine the flow rate of the bodily fluid in the flow tube from the measured temperature drop of the heating element over the predetermined amount of cooling time and a curve fit to a stored set of previously obtained calibration measurements. An implant power and communication subsystem coupled to the implant microcontroller is configured to wirelessly receive power and wirelessly transmit and receive data. The system also includes an external device having an external microcontroller, and an external power and communication subsystem coupled to the external microcontroller configured to wirelessly deliver power to the implant power and communication subsystem and transmit and receive data to and from the implant power and communication subsystem.

In another aspect, a flow rate sensor system for non-invasively measuring the flow rate of a bodily fluid is featured. The system includes an encapsulated implant having a heating element externally coupled to a shunt, catheter, tube, or vessel configured to receive a flow of a bodily fluid, the heating element configured to dissipate heat at a predetermined rate over a predetermined amount of time. A temperature sensor externally coupled to the heating element is configured to measure a temperature rise of the heating element over the predetermined amount of time. An implant microcontroller coupled to the temperature sensor is configured to determine the flow rate of the bodily fluid in the shunt, catheter, tube or vessel from the measured temperature rise of the heating element over the predetermined amount of time and a curve fit to a stored set of previously obtained calibration measurements. An implant power and communication subsystem coupled to the implant microcontroller is configured to wirelessly receive power and wirelessly transmit and receive data. The system also includes an external device having an external microcontroller and an external power and communication subsystem coupled to the external microcontroller configured to wirelessly deliver power to the implant power and communication subsystem and transmit and receive data to and from the implant power and communication subsystem.

In one embodiment, the encapsulated implant may be configured as a two-piece clamp externally coupled to the shunt, catheter, tube, or vessel.

In another aspect, a flow rate sensor system for non-invasively measuring the flow rate of a bodily fluid is featured. The system includes an encapsulated implant having a heating element externally coupled to the shunt, catheter, tube, or vessel configured to receive a flow of a bodily fluid, the heating element configured to dissipate . heat at a predetermined rate over a predetermined temperature rise of heating element. A temperature sensor externally coupled to the heating element is configured to measure a temperature drop of the heating element over a predetermined amount of time of cooling. An implant microcontroller coupled to the temperature sensor is configured to determine the flow rate of the bodily fluid in the shunt, catheter, tube or vessel from the measured temperature drop of the heating element over the predetermined amount of cooling time and a curve fit to a stored set of previously obtained calibration measurements. An implant power and communication subsystem coupled to the implant microcontroller is configured to wirelessly receive power and wirelessly transmit and receive data. The system also includes an external device having an external microcontroller and an external power and communication subsystem coupled to the external microcontroller configured to wirelessly deliver power to the implant power and communication subsystem and transmit and receive data to and from the implant power and communication subsystem.

In one embodiment, the encapsulated implant may be configured as a two-piece clamp externally coupled to the shunt, catheter, tube, or vessel.

In another aspect, a method for non-invasively measuring the flow rate of a bodily fluid is featured. The method includes providing an encapsulated implant coupled to a shunt, catheter, tube or vessel, receiving a flow of a bodily fluid in the shunt, catheter, tube or vessel, externally coupling a heating element to the shunt, catheter, tube or vessel configured to dissipate heat at a predetermined rate over a predetermined amount of time, externally coupling a temperature sensor to the heating element, measuring a temperature rise of the heating element over a predetermined amount of time, determining the flow rate of the bodily fluid in the shunt, catheter, tube or vessel from the measured temperature rise and a curve fit to a stored set of previously obtained calibration measurements, providing an external device, wirelessly delivering power to the encapsulated implant, and wirelessly transmitting and receiving data to and from the encapsulated implant.

In one embodiment, the method may include thermally isolating the heating element and the temperature sensor. The method may further include locating the encapsulated implant using data wirelessly sent from the encapsulated implant to the external device. The method may further include positioning an external coil of the external device proximate and in alignment with an implant coil of the encapsulated implant to provide sufficient inductive coupling between an external coil of the external device and an implant coil. The method may include storing on a microcontroller of the encapsulated implant the set of previously obtained calibration measurements of heat dissipation. The method may include storing on a microcontroller of the encapsulated implant identification information associated with the encapsulated implant. The method may include determining the flow rate from a current measured temperature rise up when the temperature of the heating element is determined to be no longer rising to minimize the length of time needed to determine the flow rate, the amount of heat generated by the heating device, and the amount of heat delivered to a patient.

In another aspect a method for non-invasively measuring the flow rate of a bodily fluid is featured. The method includes providing an encapsulated implant coupled to a shunt, catheter, tube or vessel, receiving a flow of a bodily fluid in the a shunt, catheter, tube or vessel, externally coupling a heating element to the shunt, catheter, tube or vessel configured to dissipate heat until a predetermined rate temperature rise is achieved, externally coupling a temperature sensor to the heating element, measuring a temperature drop of the heating element over a predetermined amount of time of cooling, determining the flow rate of the bodily fluid in the flow tube from the measured temperature drop and a curve fit to a set of previously obtained calibration measurements, providing an external device, wirelessly delivering power from the external device to the encapsulated implant, and wirelessly transmitting and receiving data to and from the encapsulated implant and the external device.

### BRIEF DESCRIPTION OF THE SEVERAL VIEWS OF THE DRAWINGS

Other objects, features and advantages will occur to those skilled in the art from the following description of a preferred embodiment and the accompanying drawings, in which:

FIG. 1 is a perspective side view showing the primary components of one embodiment of the flow rate sensor system and method thereof for non-invasively measuring the flow rate of a bodily fluid through a shunt implanted in a patient.

FIG. 2 is a top-view showing in further detail one embodiment of the encapsulated implant shown in FIG. 1;

FIG. 3 is a top-view of the encapsulated implant shown in FIG. 2 without the encapsulant thereon;

FIG. 4 is a schematic end-view showing in further detail one example of the placement of the heating element and temperature sensor about the flow tube shown in FIGS. 2 and 3;

FIG. 5 is a graph depicting one example of a stored set of previously obtained calibration measurements showing a relationship between the temperature rise of the heating element and the flow rate of the bodily fluid used by the encapsulated implant 12 shown in one or more of FIGS. 1-3;

FIG. 6 is a graph depicting one example of a curve fit to the previously obtained calibration measurements shown in FIG. 5;

FIG. 7 is a graph showing one example of the flow rate measured by the encapsulated implant shown in one or more of FIGS. 1-3 compared to the actual flow rate imposed by a syringe pump;

FIG. 8 is a graph depicting another example of a stored set of previously obtained calibration measurements showing a relationship between the temperature drop of a heating element and the flow rate of the bodily fluid used by the encapsulated implant 12 shown in one or more of FIGS. 1-3;

FIG. 9 is a graph depicting one example of a curve fit to the previously obtained calibration measurements shown in FIG. 8;

FIG. 10 is a schematic block diagram showing one embodiment of the primary components of the implant power and communication subsystem of the encapsulated implant shown in one or more of FIGS. 1-3;

FIG. 11 is a schematic block diagram showing one embodiment of the primary components of the external power and communication subsystem of the external device shown in FIG. 1;

FIG. 12 shows an example of a resistance temperature detector (RTD) which may be used for the temperature sensor shown in at least FIGS. 3, 4, and 10;

FIG. 13 shows an example of a thermocouple which may be used for the temperature sensor shown in at least FIGS. 3, 4, and 10;

FIG. 14 shows an example of the heating element configured as a coil of electrically conductive wire;

FIG. 15 shows an example of the heating element configured as a printed circuit heater a resistor;

FIGS. 16-17 show examples of the heating element configured as a resistor;

FIG. 18 is a schematic end-view showing one example of an insulation layer which may be placed about the heating element and temperature sensor shown in at least FIGS. 3, 4, and 10;

FIG. 19 is a schematic end-view showing an example of an insulation layer of sealed air surrounding the heating element and temperature sensor shown in FIGS. 3, 4, and 10;

FIG. 20 is a three-dimensional view showing in further detail the insulation layer of sealed air surrounding the heating element and temperature sensor shown in FIG. 19;

FIG. 21 is a front side-view showing one example of the primary components of a VP shunt and various locations of the encapsulated implant on a ventricular catheter or a distal catheter;

FIG. 22 is a side-view showing in further detail one example of the primary components of the flow rate sensor system shown in one or more of FIGS. 1-19;

FIG. 23 is a front side-view showing in further detail one example of the alignment of the implant coil shown in at least FIGS. 2, 3, and 10 with the external coil of the external power and communication subsystem shown in at least FIGS. 1 and 22;

FIG. 24 is a schematic diagram showing one example of the implant coil separately located from the encapsulated implant; and

FIG. 25 is a three-dimensional front-view showing the primary components of another embodiment of the flow rate sensor system for non-invasively measuring the flow rate of a bodily fluid through a shunt catheter, tube or vessel.

### DETAILED DESCRIPTION OF THE INVENTION

Aside from the preferred embodiment or embodiments disclosed below, this invention is capable of other embodiments and of being practiced or being carried out in various ways. Thus, it is to be understood that the invention is not limited in its application to the details of construction and the arrangements of components set forth in the following description or illustrated in the drawings. If only one embodiment is described herein, the claims hereof are not to be limited to that embodiment. Moreover, the claims hereof are not to be read restrictively unless there is clear and convincing evidence manifesting a certain exclusion, restriction, or disclaimer.

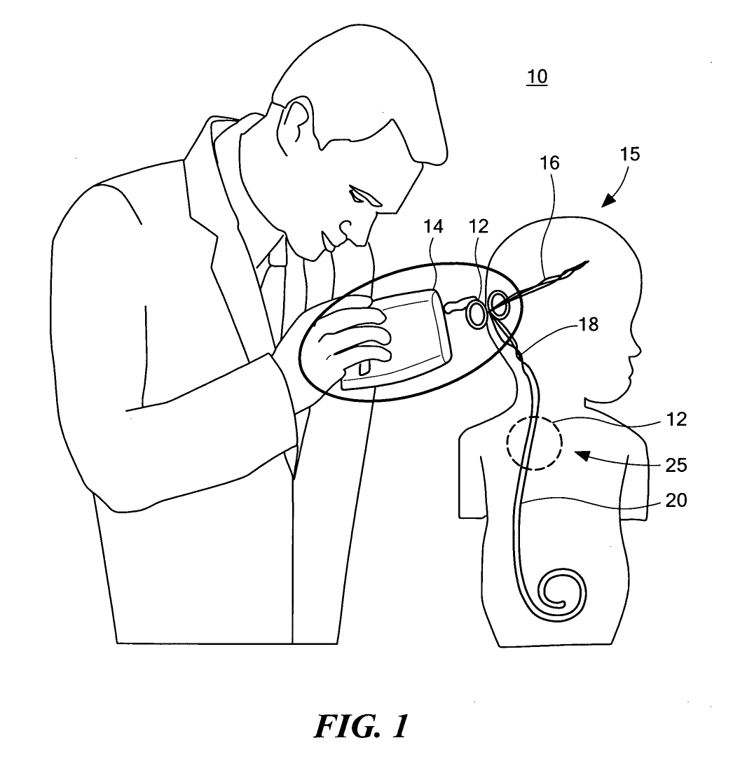

There is shown in FIG. 1 one embodiment of system 10 and the method thereof for non-invasively measuring the flow rate of a bodily fluid. System 10 includes encapsulated implant 12 and external device 14. In the example shown in FIG. 1, encapsulated implant 12 is implanted into human body 15 and is coupled to ventricular or proximal catheter 16 of VP shunt 18. In other examples, encapsulated implant 12 (shown in phantom) may be coupled to distal catheter 20 of VP shunt 18, indicated at 25. However, encapsulated implant 12 need not necessarily be coupled in-line to VP shunt 18 as shown and may be externally coupled over any type catheter, shunt, tube, vessel and the like, which is implanted in the human body or the body of an animal and has a flow of bodily fluid there through, as discussed in further detail below.

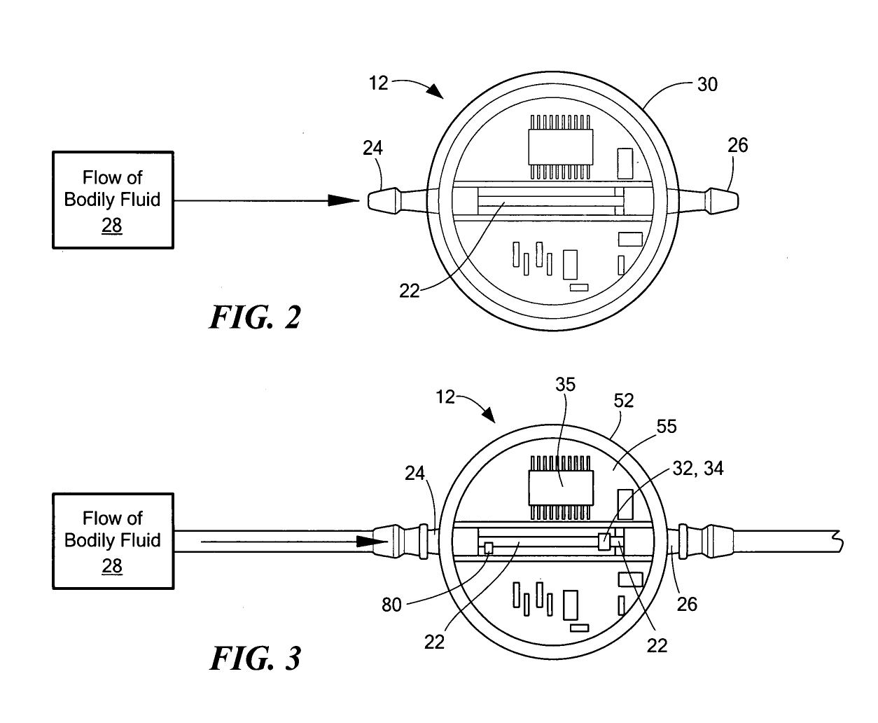

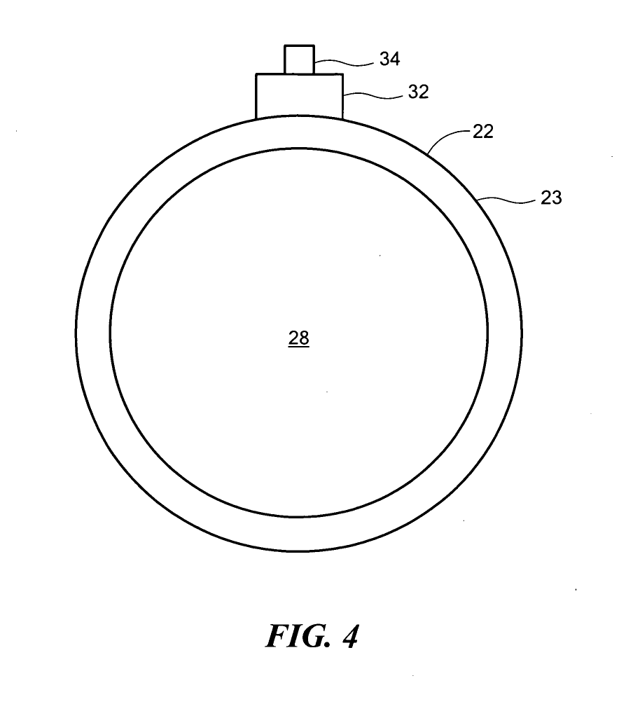

In this example, encapsulated implant 12, FIG. 2, includes flow tube 22 having inlet 24 and outlet 26 configured to receive flow of bodily fluid 28. Flow of bodily fluid 28 may include CSF, blood, bile, urine, or other bodily fluid. Encapsulated implant 12 includes encapsulant 30, e.g., a medical grade polyurethane such as Steralloy FDF 2380 (Hapco, Inc. Hanover, Mass. 02339), silicone, or other bio-compatible materials. Encapsulated implant 12, FIG. 3, where like parts have been given like numbers, shown without encapsulant 30 for clarity, also includes heating element 32 externally coupled to flow tube 22 and temperature sensor 34 externally coupled to heating element 32. FIG. 4 shows in further detail one example of heating element 32 directly and externally coupled to external surface 23 of flow tube 22 and temperature sensor 34 externally coupled to heating element 32. In one embodiment, heating element 32, FIGS. 3 and 4, is configured to dissipate heat at a predetermined rate over a predetermined amount of time. In this example, temperature sensor 34 is configured to measure the temperature rise of heating element 32 over a predetermined amount of time, e.g., 10 seconds, 20 seconds, 30 seconds, and the like, as discussed in further detail below.

Encapsulated implant 12, FIG. 3, also includes implant microcontroller 35, preferably coupled to printed circuit board (PCB) 55, configured to determine the flow rate of flow of bodily fluid 28 in flow tube 22 from the temperature rise of heating element 32 over the predetermined amount of time and a curve fit to a stored set of previously obtained calibration measurements. The stored set of previously obtained calibration measurements include measurements of the temperature rises of same heating element 32 and associated imposed flow rates over the same predetermined amount of time and level of heat dissipation. The store set of previously obtained calibration measurements are preferably stored by implant microcontroller 35.

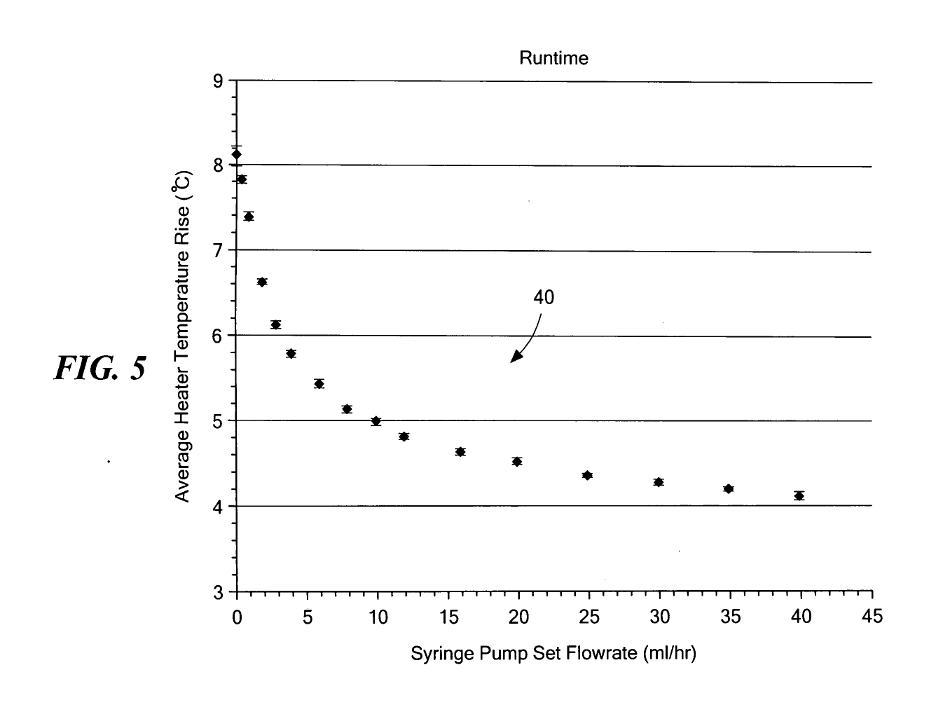

In one example, the stored set of previously obtained calibration measurements shown by data points 40, FIG. 5, may be created by a user request using external device 14, FIG. 1. The stored set of previously obtained measurements may be created by applying a regulated DC voltage to heating element 32, FIGS. 3 and 4, that produces a repeatable heat dissipation level from the heating element 32 each time the regulated DC voltage is applied, discussed in further detail below. The resulting heat created within heating element 32 causes a rise in temperature of the heating element 32 over the same predetermined amount of time, e.g., 5, 10, 15, 20, 30, or 40 seconds, or similar time intervals, as that used for the flow rate measurement. The rise in temperature of heating element 32 is measured by temperature sensor 34 over the predetermined amount of time, while heating element 32 is turned on and dissipating heat. In order to accurately obtain the temperature rise of temperature sensor 34 and heating element 32, temperature measurements are recorded for a few seconds prior to turning on heating element 32. The average of these temperature measurements may serve as a baseline temperature that can be subtracted from the temperature measurements taken after heating element 32 is turned on.

To create the stored set of previously obtained calibration measurements, the resulting temperature rise over the predetermined amount of time is preferably matched to the imposed flow rate, and two values are stored in the memory of the implant microcontroller 35 as the stored set of calibration measurements. In one design, rather than store only one value of temperature rise for calibration, multiple values of temperature rise versus flow rate over the predetermined amount time and rate of heat dissipation may be stored.

In one example, a pump capable of accurately delivering a known desired flow rate, such as a well-calibrated syringe pump may be used to create the stored calibrated measurement. The stored set of calibrated flow rate measurements may be obtained in this manner at each of multiple flow rate settings over the known range of feasible bodily fluid flow rates through flow tube 22, e.g., from about 0 to about 40 mL/hr.

The number of calibration values for the stored set of calibrated flow rate measurements is preferably sufficient to characterize a curve of the rise in temperature of heating element 32 as a function of flow rate, e.g., data points 40, FIG. 5. The heat dissipation from heating element 32, FIGS. 3 and 4, need not be precisely known, but is preferably repeatable each time heating element 32 and encapsulated implant 12, FIGS. 1-3, is activated.

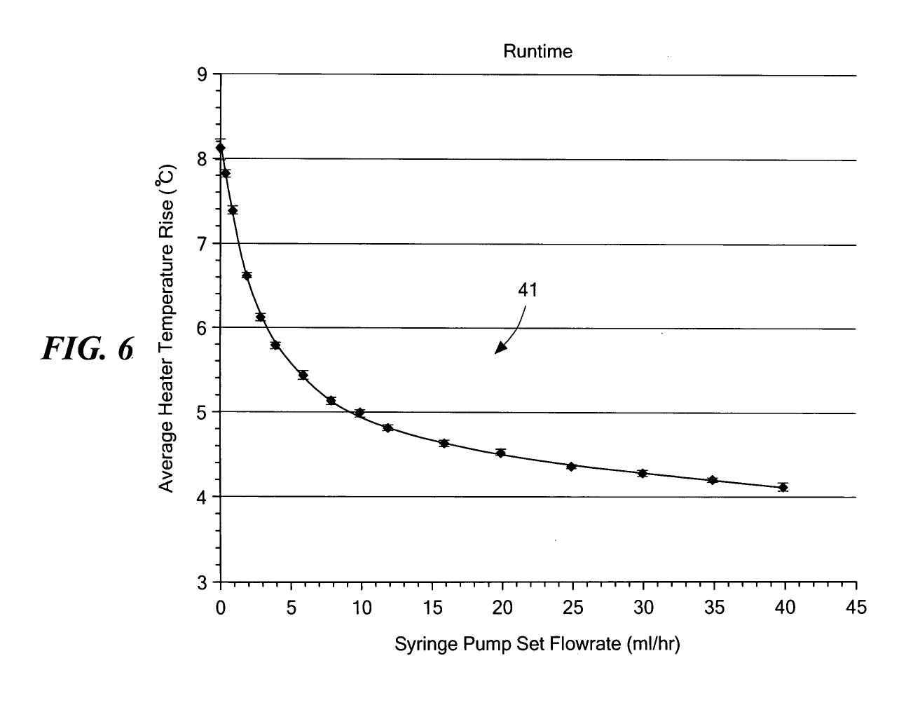

In operation, a regulated DC voltage is applied to heating element 32 and the temperature rise of heating element 32 is sensed by temperature sensor 34 and recorded by implant microcontroller 35. Curve fitting is preferably applied to the stored set preferably previously obtained calibration measurements of temperature rise versus flow rate to derive a continuous relationship between measured temperature rise and flow rate, as shown by curve 41, FIG. 6. From curve 41 and the measured temperature rise, the flow rate of flow of bodily fluid 28, FIG. 3, through flow tube 22 over the duration of the measurement is inferred.

Curve fitting is a well understood process of creating a curve or a continuous mathematical function that closely fits a series of data points. For determination of flow rate, curve fitting can involve either interpolation between calibration data points of measured temperature rises versus flow rates, or the determination of a smooth mathematical function that fits all the data points 40, FIG. 5, to a good approximation. Curve 41, FIG. 6, shows one example of curve fitting in which linear extrapolation is employed between each of the adjacent data points 40 shown in FIG. 5.

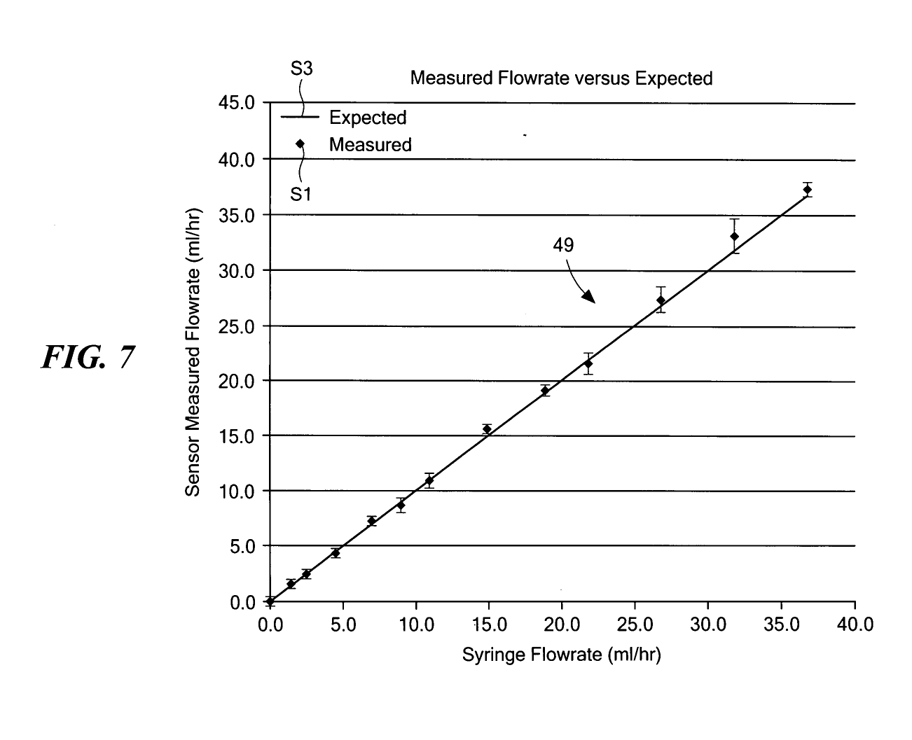

Plot 49, FIG. 7, shows one example of the flow rates measured by encapsulated implant 12, indicated at 51, compared to actual known flow rates imposed by a calibrated syringe pump, indicated at 53. As can be seen, encapsulated implant 12, FIGS. 1-3, accurately determined the flow rate of flow of bodily fluid 28 in flow tube 22.

If the implant microcontroller 35 determines that the temperature of heating element 32 is no longer rising (i.e. that steady state has been reach), then implant microcontroller 35 can terminate the measurement since it has already acquired a sufficient number of temperature values from temperature sensor 34 to determine the flow rate. This can reduce the predetermined amount of time needed to determine the flow rate of flow of bodily fluid 28, e.g., to between about 5 to 10 seconds and minimize the amount of heat needed to be generated by heating element 32.

The dependence of the temperature rise of heating element 32 over the duration of a flow rate measurement by encapsulated implant 12 rate arises from the flow rate of flow of bodily fluid 28, FIG. 2, and the heat transfer coefficient for heat transfer from heating element 32 into flow of bodily fluid 28 inside tube 22 in accordance with formula:

h=Q/(AΔT) (1)

where Q is the heat dissipated by the heating element 32, A is the area of heat transfer, and AT is the difference in temperatures between heating element 32 and bodily fluid 28 flowing in tube 22. The heat transfer coefficient h increases monotonically with flow rate, at least over the range of CSF flow rates possible within a shunt. Therefore, since the level of heat dissipation Q produced by the heating element is fixed for a given applied DC voltage, the temperature rise of heating element 32 during a flow rate measurement will decrease with increasing CSF flow rate.

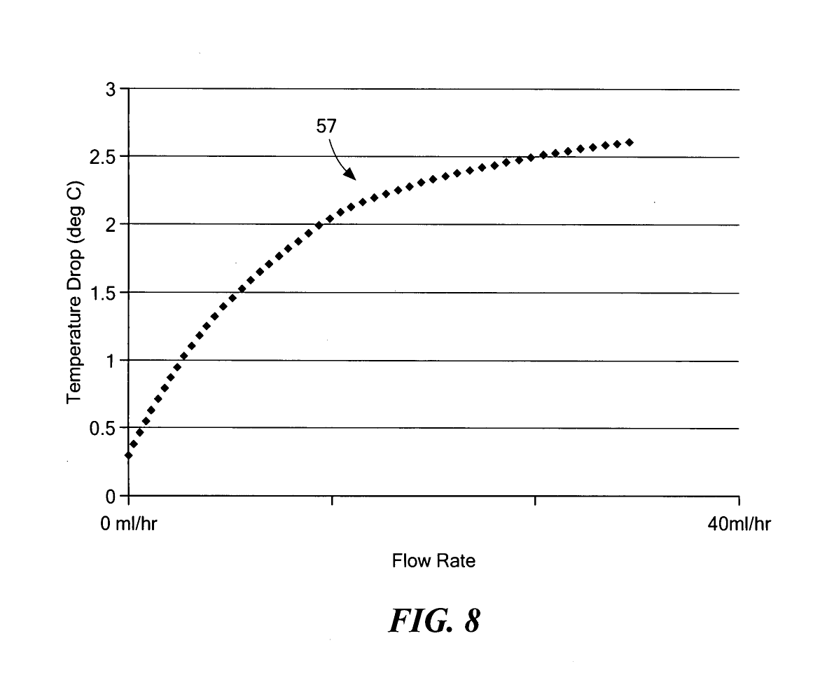

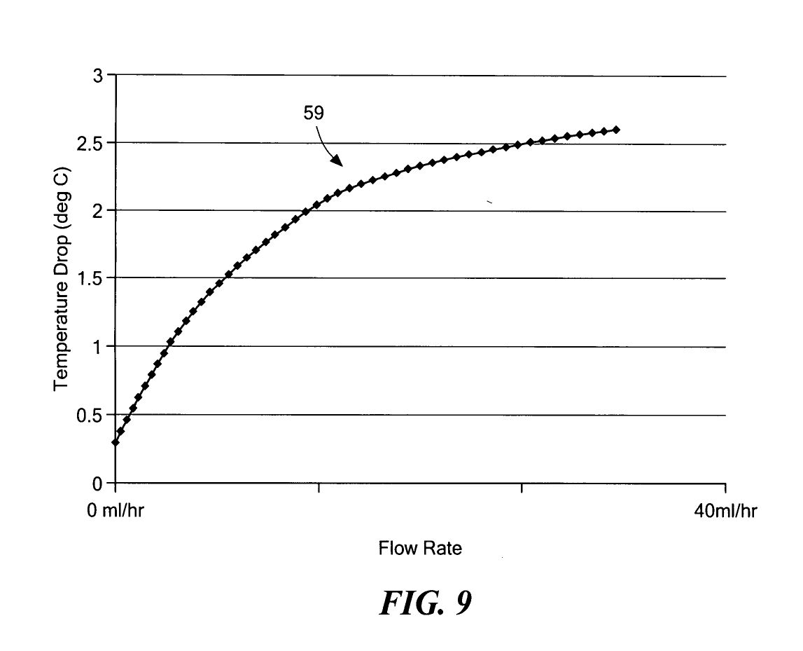

Flow rate sensor system 10, shown in one or more of FIGS. 1-4, may also be used to determine flow rate by measuring the temperature drop of heating element 32 after it is allowed to cool for a predetermined amount of time, e.g., 5, 10, 20, 30 seconds, and the like, after the heating element 32 has been previously heated until a predetermined temperature rise is achieved, e.g., 2, 3, 4° C., and the like. The measured temperature drop is then compared with a set of previously obtained calibration measurements in which temperature drops were measured and stored after heating element 32 was first heated to the same temperature rise and then turned off for the same predetermined amount of time, while precisely known flow rates were imposed. Similarly, as discussed above with reference to FIGS. 5 and 6, a curve fit or interpolation may be used to estimate flow rate from the stored set of previously obtained calibration measurements. For example, a flow rate may be determined by first turning on heating element 32 and allowing it to continue to warm up until implant microcontroller 35, FIG. 3, detects a predetermined rise in temperature has been achieved, e.g., about 3° C. At this point heating element 32 is turned off and allowed to cool for a predetermined period of time e.g., 5, 10, 15 seconds, and the like. During the cooling of heating element 32, the temperature drop of heating element 32 over a predetermined amount of time will depend on the flow rate of bodily fluid 28 in tube 22, since the rate of cooling of heating element 32 depends on the flow rate of bodily fluid 28. In particular, the rate of cooling will increase as the flow rate of bodily fluid 28 increases, and, therefore, the temperature drop of heating element 32 over a predetermined amount of time of cooling will also increase as flow rate of bodily fluid 28 increases. In this example, implant microcontroller 35 is configured to determine the flow rate of the bodily fluid in the flow tube from the measured temperature drop of the heating element 32 and curve fit to a stored set of previously obtained calibration measurements having a relationship between temperature drop of heating element and flow rate of bodily fluid established similar as described above. In this example, data points 57, FIG. 8, is utilized instead of data points 40, FIG. 5 described above. Curve-fitting or interpolation is preferably applied to the set of previously obtained calibration measurements shown in FIG. 8 in order to infer the flow rate through tube 22 over the duration of the measurement, e.g. as shown by curve 59, FIG. 9.

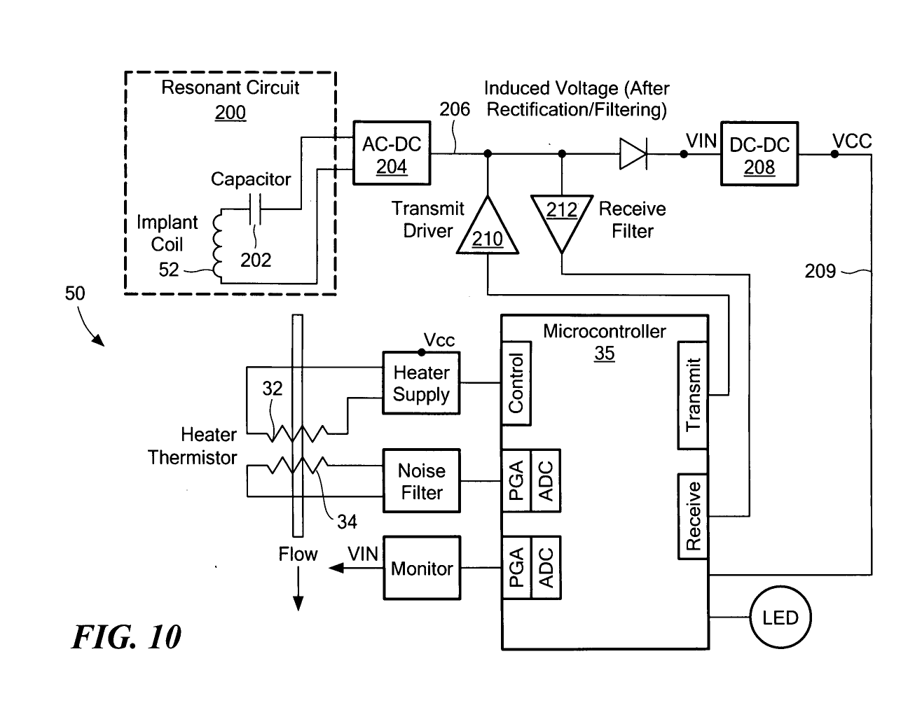

Encapsulated implant, FIGS. 1-3, also includes implant power and communication subsystem 50, FIG. 10, preferably formed on printed circuit board 55, FIG. 3. Implant power and communication subsystem 50, FIG. 10, is configured to wirelessly receive power and transmit and receive data.

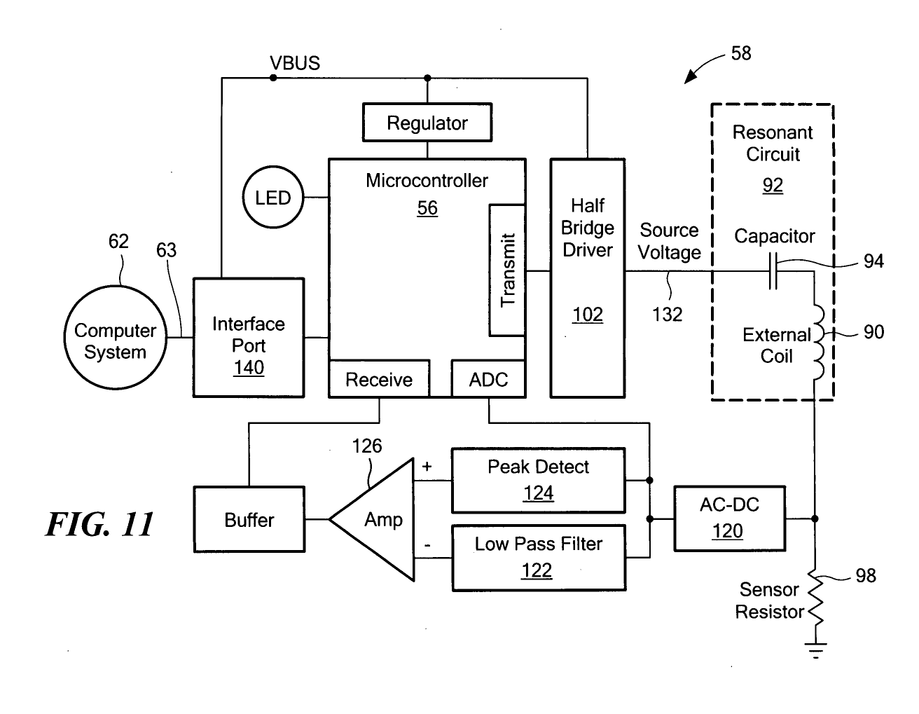

External device 14, FIG. 1, of system 10 also includes external microcontroller 56, FIG. 11, and external power and communication subsystem 58 coupled to external microcontroller 56 configured to wirelessly deliver power to implant power and communication subsystem 50, FIG. 10, and wirelessly transmit and receive data to and from implant power and communication subsystem 50 of encapsulated implant 12, FIGS. 1-3, as discussed in further detail below.

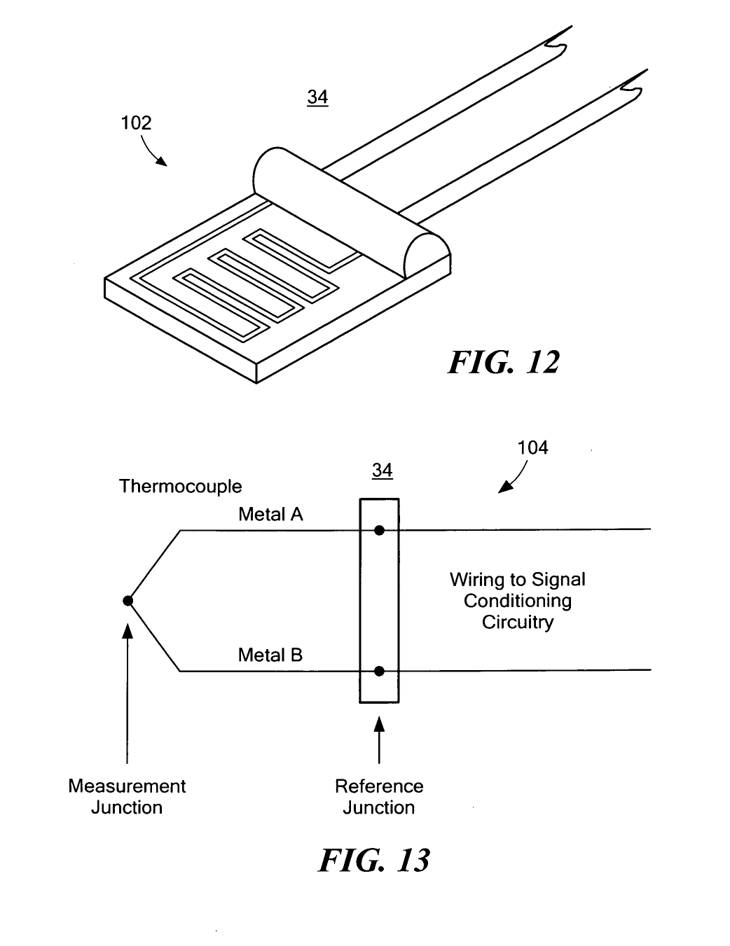

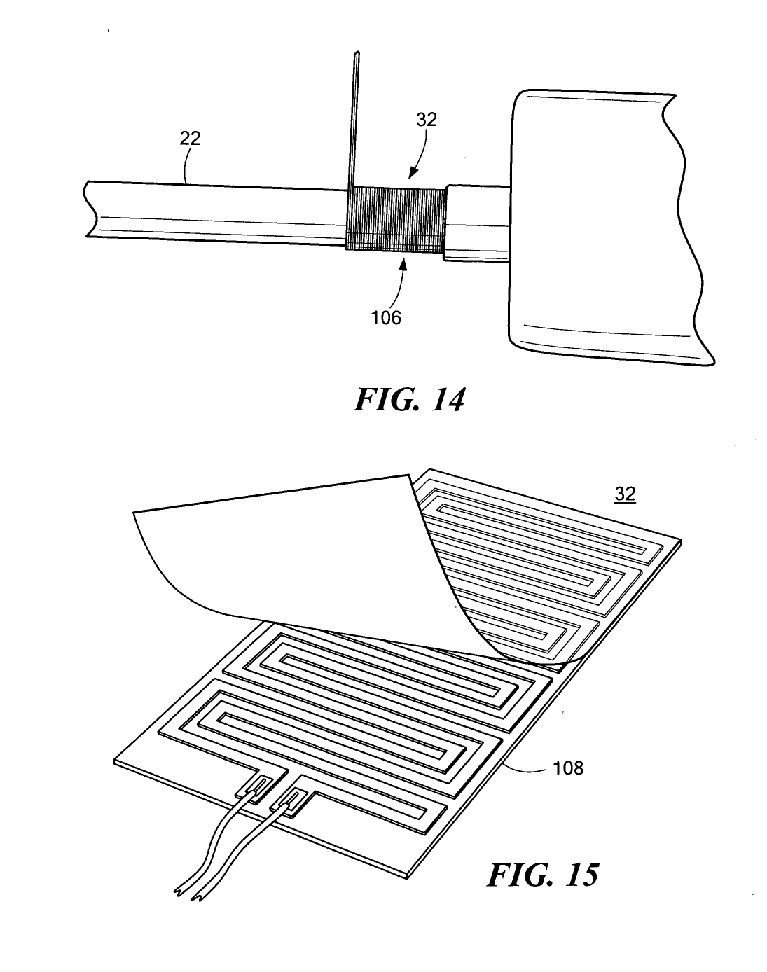

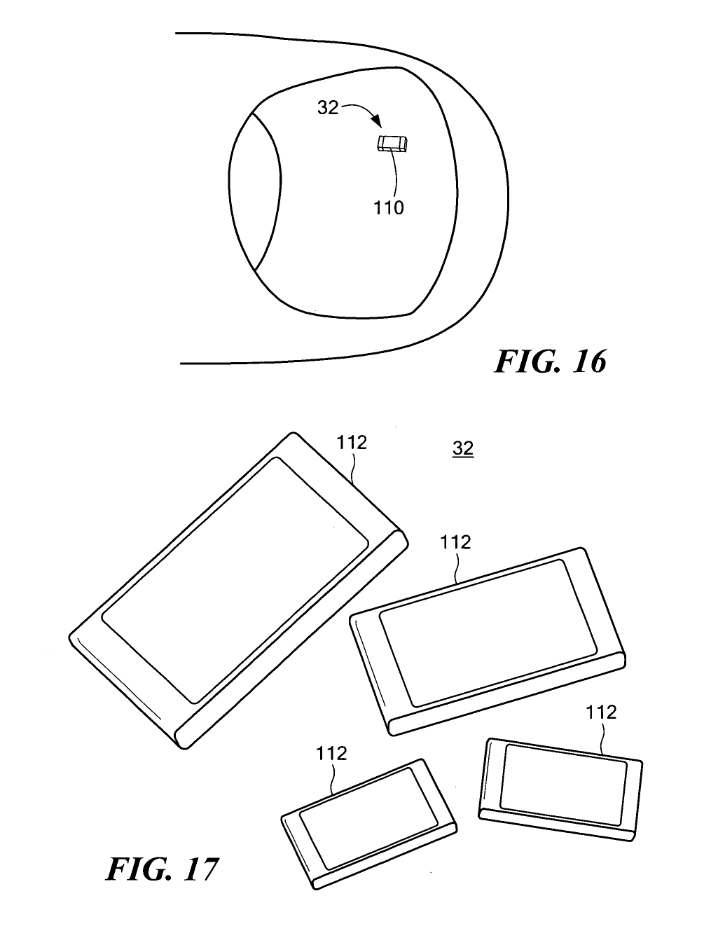

In one example, temperature sensor 34, FIGS. 3 and 4, includes a thermistor, e.g., thermistor 34′ as shown in FIG. 10. In other designs, temperature sensor 34 may be a resistance temperature detector (RTD), e.g. RTD 102, FIG. 12, or a thermocouple, e.g., thermocouple 104, FIG. 13. In other examples, heating element 32 may be a coil of electrically conductive wire wound around the flow tube 22, e.g., coil 106, FIG. 14, of electrically conductive wire. In another design, heating element 32 may be a printed circuit heater, e.g., printed circuit heater 108, FIG. 15. In yet another design, heating element 32 may be a resistor (either surface mount or leaded), e.g. resistor 110, FIG. 16, or any of resistors 112, FIG. 17.

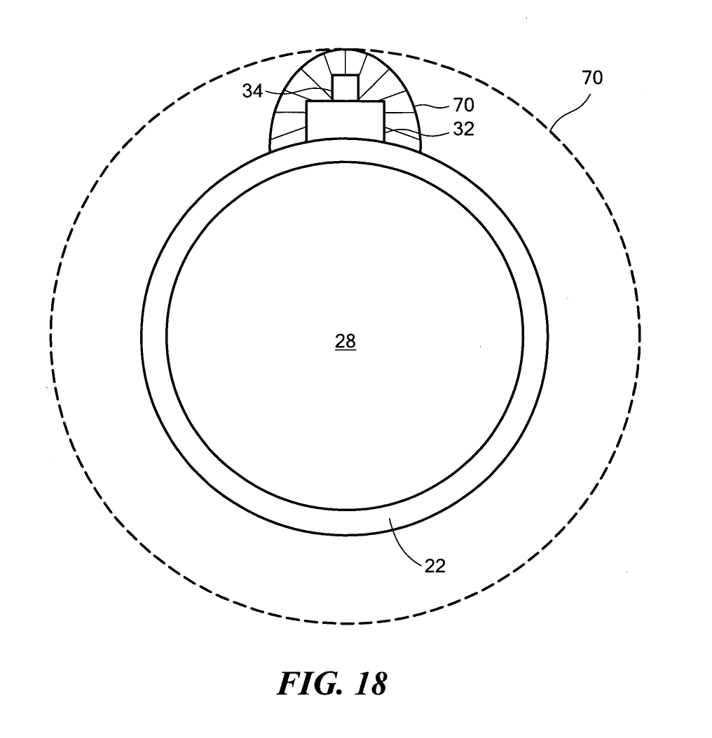

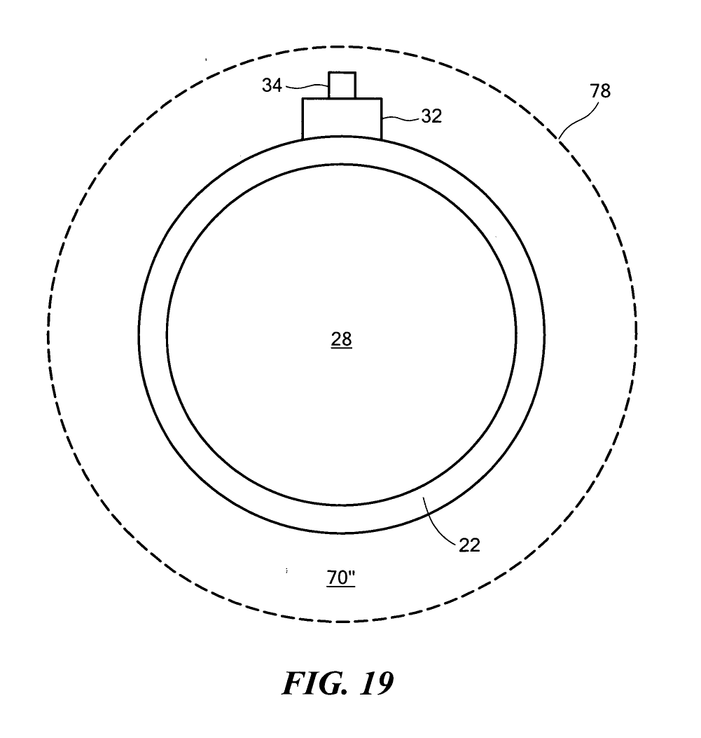

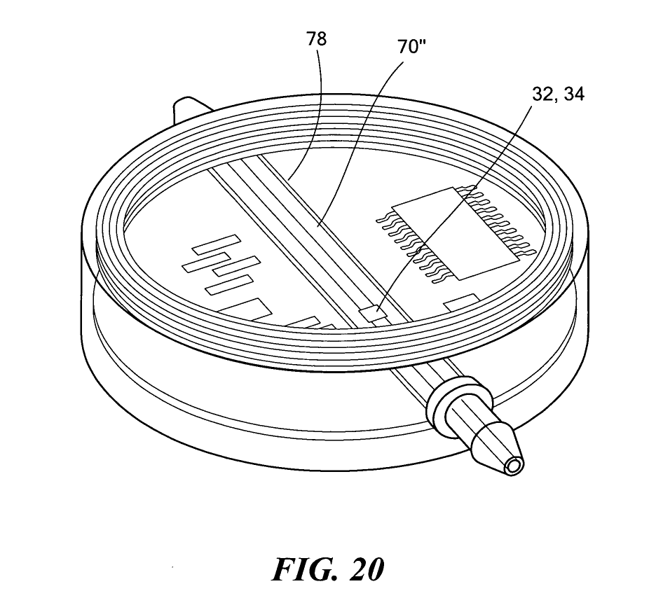

Preferably, heating element 32, FIGS. 3 and 4, is directly attached to flow tube 22 as shown in FIG. 4. The rise in temperature of the heating element 32 over the predetermined amount of time or the temperature drop over the predetermined amount of time of cooling represents the ‘signal’ employed during a flow rate measurement to determine flow rate. The signal may be increased by thermally isolating the heating element 32 from heat transfer paths other than conduction/convection to the flow of bodily fluid 28, FIG. 3, flowing through the flow tube 22. In one design, encapsulated implant 12 includes thermal insulator 70, FIG. 18, which preferably covers heating element 32 and temperature sensor 34 as shown to thermally isolate heating element 32 and temperature sensor 34 from heat transfer paths other than conduction/convection to the flow of bodily fluid 28, FIG. 3. Thermal insulator 70 may also surround all of flow tube 22, as shown by thermal insulator 70′ in phantom. In other examples, insulation layer 70″, FIG. 19, may be a pocket of sealed air created by surrounding flow tube 22 with hollow tube 78. FIG. 20, where like parts have been given like numbers, shows in further detail insulation layer 70″ of sealed air and hollow tube 78 surrounding heating element 32 and temperature sensor 34 and flow tube 22.

In one example, flow tube 22, FIGS. 3, 4, 18-20, may be made of a thin walled polymer material with low thermal conductivity, such as polyimide or similar type material, to limit heat transfer along the length and circumference of flow tube 22 while maintaining heat transfer in the radial direction to the bodily fluid in tube 22 made viable by the thin wall thickness of the tube.

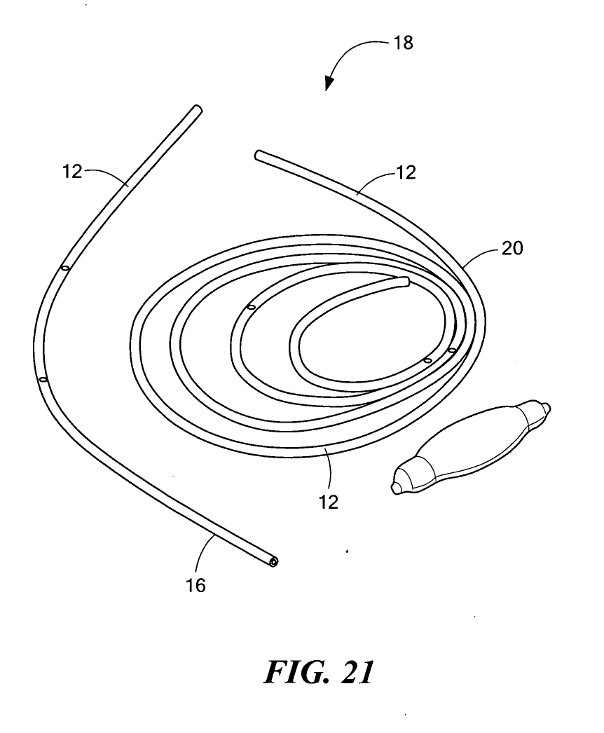

As discussed above, encapsulated implant 12, FIG. 1, may be coupled to VP shunt 18, e.g., to distal catheter 20 of VP shunt 18 or proximal catheter 16. FIG. 21 shows in further detail one example of the structure of VP shunt 18 with distal catheter 20 and proximal or ventricular catheter 16. Encapsulated implant 12 may be located at any position on ventricular catheter 16 or distal catheter 20 as shown. In other examples, encapsulated implant may be coupled to shunt, catheter, tube, or vessel implanted in the body, such as a ventroarterial shunt or a lumboperitoneal shunt.

In the example shown in FIGS. 2 and 3, heating element 32 and temperature sensor 34 are shown located proximate outlet 26. In other examples, heating element 32 and temperature sensor 34 may be located proximate inlet 24 indicated at 80, FIGS. 3, or between inlet 24, and outlet 26, as indicated at 82.

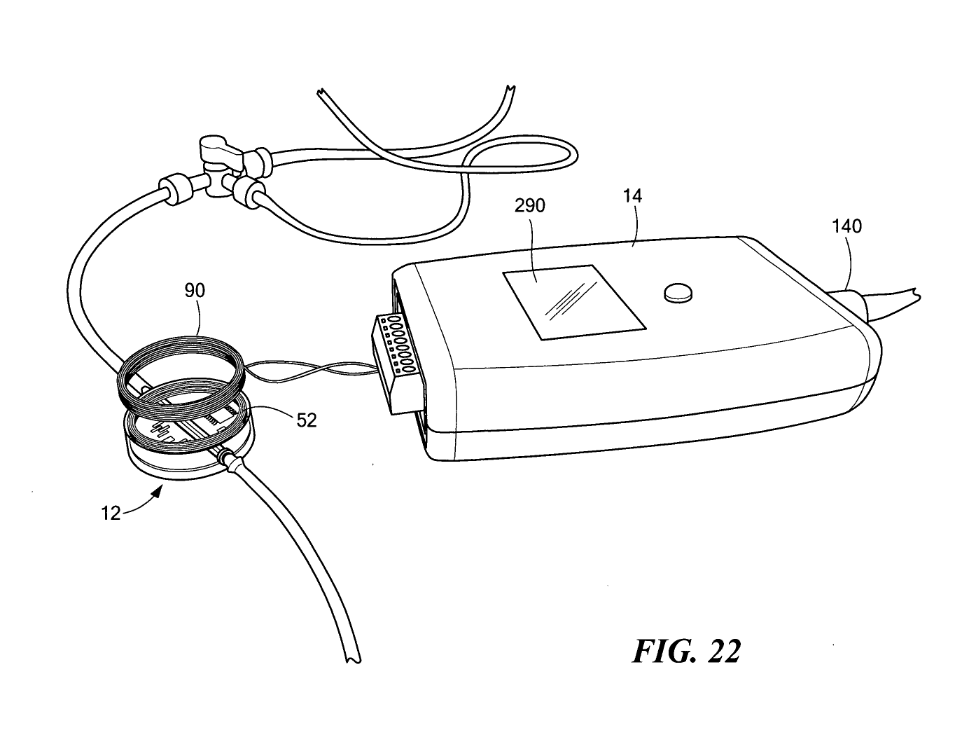

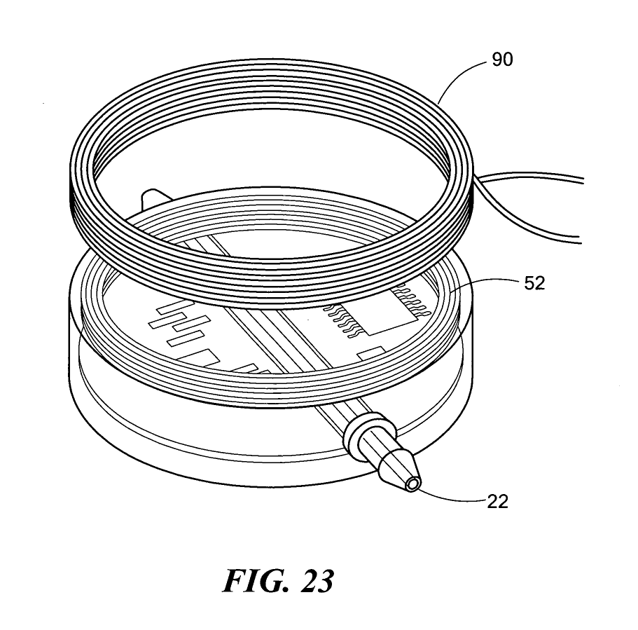

External power and communication subsystem 58, FIG. 11, of external device 14, FIG. 1, includes external coil 90, FIG. 11 coupled to microcontroller 56. FIG. 22, where like parts have been given like numbers, shows in further detail one example of external coil 90 of external device 14 shown placed in close proximity to implant coil 52 of encapsulated implant 12. As shown, implant coil 52 is integrated with encapsulated implant 12, as depicted in further detail in FIG. 3. External power and communication subsystem 58, FIG. 11, is configured to inductively transfer power from external coil 90, FIGS. 11 and 22, to implant coil 52, FIGS. 3 and 22, of implant power and communication subsystem 50, FIG. 10.

It is well known that the presence of a time-varying current in one coil will induce a voltage in a nearby second coil. This principle is employed by system 10 for non-invasively measuring the flow rate of a bodily fluid to enable wireless power transfer and communication between external device 14 and encapsulated implant 12. The voltage (V₂) induced in the implant coil 52 by external coil 90 may be shown by the equation:

V₂(t)=M(dI₁/dt) (2)

where M is the mutual inductance between the implant coil 52 and external coil 90 and I₁ (t) is the current in the external coil 90. If the current in the external coil 90 coil is sinusoidally-varying in time at a frequency ω=2πf, where f is the frequency in Hertz, then:

V₂=ωMI₁, (3)

where V₂ and I₁ are the amplitude of the voltage induced in implant coil 52 and the amplitude of the current in the external coil 90, respectively. Likewise, if the current in the implant coil is time-varying, a voltage is induced in external coil 90 given by:

V₁=ωMI₂, (4)

where V₁ and I₂ are the amplitude of the voltage induced in the external coil 90 and the amplitude of the current in external coil 90, respectively

The mutual inductance depends both on the self-inductances of the coupled external coil 90 (L₁) and implant coil 52 (L₂) coils and the coupling coefficient (K_{C}) between them:

M=K_{c}(L₁L₂)^{1/2}, (5)

where K_{C} depends on relative orientation, lateral alignment and proximity of the external coil 90 and implant coil 52. The self-inductance of the external coil 90 (L₁) is preferably set such that the source voltage 132, FIG. 11, of the external power and communication subsystem 58 is at a convenient and safe level, whereas the self-inductance of implant coil 52 (L₂) and the coupling coefficient (K_{C}) are preferably sufficient such that the induced voltage (after rectification and filtering) on implant power and communication subsystem 50, FIG. 10, of encapsulated implant 12 is high enough to meet the input voltage specifications of the DC-DC converter 208, FIG. 10, that provides the regulated

DC voltage necessary to operate the implant microcontroller 35, heating element 32, temperature sensor 34 and other various electronic components of encapsulated implant 12, FIG. 3, e.g., printed circuit board (PCB) 55 and the various electronics thereon.

External power and communication subsystem 58, FIG. 11, preferably includes external resonance circuit 92 comprised of external coil 90, and capacitor 94, and source voltage 132 generated by a half bridge driver 103 or by other equivalent device known to those skilled in the art. The external power and communication subsystem 58 is preferably configured to generate AC current flow at a predetermined resonance frequency in external coil 90, in order to induce sinusoidal voltage signals in implant coil 52, shown in at least FIGS. 10 and 22.

In one example, resonant circuit 92, FIG. 11, and analog electronics 96 filter and amplify changes in the voltage drop across current sense resistor 98 in order to recover data communications bits transmitted from implant power and communication subsystem 50, FIG. 10, of encapsulated implant 12. Half-bridge driver circuit 103, FIG. 11, with a dedicated controller and two MOSFETs (not shown) may be used to drive external coil 90. External coil 90 in combination with series capacitor 94 preferably forms a resonant circuit with a predetermined resonant frequency, e.g., 100 kHz. In one example, a 100 kHz square wave generated by external microcontroller 56 may be applied to half-bridge driver circuit 103 to create a sinusoidal current flow through the external coil 90. The frequency may be adjusted to produce the closest match between resonant circuit 92 and resonant circuit 200, FIG. 10, of implant power and communication subsystem 50, as discussed below. The voltage drop across sense resistor 98, FIG. 11, may be used to monitor the current through external coil 90. The voltage across sense resistor 98 is preferably converted to DC by AC-DC rectifier 120 and filtered by filter 122 and peak detector 124 to remove the 100 kHz signal. The difference between the peak voltage and the filtered voltage is then amplified by amplifier 126, converted to digital signal levels and fed to external microcontroller 56 for decoding of the digital data transmitted by the implant power and communication subsystem 50, FIG. 10 of encapsulated implant 12. In order to provide communications as well as power, external power and communication subsystem 58, FIG. 11, of external device 14 can modulate the square wave signal delivered to half-bridge driver 103 to encode information. The implant power and communication system 50, FIG. 10, can decode the modulation in order to recover the data being transmitted.

The inductance of external coil 90, FIG. 11, and capacitor 94 create a resonant frequency in accordance with the formula:

fₙ=1/[2π(LC)^{1/2}] (6)

where L is the inductance of external coil 90 and C is the capacitance of capacitor 94. At this frequency, the reactive impedance of capacitor 94 cancels out the reactive impedance of external coil 90, and in the vicinity of this frequency, both the reactance and overall impedance of the external power and communication subsystem 58, FIG. 11, of external device 14 are greatly reduced.

The use of resonance frequency may be beneficial because square-wave pulses, which are conveniently produced by half bridge driver 103 or other AC voltage source known to those skilled in the art, give rise to sinusoidally-varying current in external power and communication subsystem 58. Further, the impedance of series resonant circuit 92 is a minimum at resonance, which maximizes the current for a given applied voltage, thereby lowering the voltages to levels as may be found in a common battery or USB interface, e.g., interface port 140. In addition, since the current through external coil 90 varies with the applied square wave frequency, the power delivered to the external coil 90 can be easily tuned by changing the square wave frequency. The value of capacitor 94 is preferably chosen such that the capacitor 94 and external coil 90 resonate at a desirable frequency. The choice of resonant frequency may include, inter alia, the available space for external coil 90, frequency-dependent coil losses, skin effect, FCC regulations, guidelines regarding patient exposure to electromagnetic fields, and the like. Preferably, resonant circuit 92 is driven by a square wave source voltage 132, with its frequency set at or near the resonant frequency of resonant circuit 92. This results in a sinusoidally-varying current in the external power and communication subsystem 58 at the frequency of the voltage source pulses. This current gives rise to a magnetic field in the space surrounding external coil 90. A fraction of the field lines of this magnetic field are inductively linked to implant coil 52, FIG. 10, thereby inducing sinusoidally-varying voltage in implant coil 52.

Implant power and communication subsystem 50, FIG. 10, of encapsulated implant 12 includes implant resonance circuit 200 comprised of implant coil 52 and capacitor 202. Implant resonance circuit 200 is preferably configured to have a resonance frequency matching the resonance frequency closely provided by resonance circuit 92, FIG. 11. The sinusoidally varying magnetic field generated by the sinusoidal current in external coil 90 links the implant coil 52 of resonant circuit 200. The resulting induced sinusoidally varying voltages in implant coil 52, FIG. 10, are then rectified by AC-DC rectifier 204 to create a DC voltage on line 206, which is applied to the input of DC-DC converter 208, which creates a constant regulated DC voltage on line 209. DC-DC power supply 208 provides power to implant microcontroller 35, heating element 32, e.g., a thermistor, in this example, acting as temperature sensor 34, and other components on PCB 55, FIG. 3, of encapsulated implant 12, which may require power.

In one example, external device 14, FIGS. 1 and 22, encodes digital data for communication with encapsulated implant 12, shown in one or more of FIGS. 1-3, 10 and 22, by changing the magnitude of the source voltage 132, FIG. 11, which gives rise to a corresponding change in current in external power and communication subsystem 58, which, in turn, gives rise to a change in the amplitude of the voltage induced on implant coil 52. Implant microcontroller 35 preferably monitors the voltage at the output of the AC-DC rectifier 204 by receive filter 212, in order to decode digital data sent from external power and communication subsystem 58 of external device 14.

Implant power and communication subsystem 50, FIG. 10, of encapsulated implant 12, FIGS. 1-3, 10 and 22, preferably communicates to external power and communication subsystem 58, FIG. 11, of external device 14, by modulating the electrical load on implant coil 52, by controlling the closure of a switch within the transmit driver 210, FIG. 10, coupled to the output of AC-DC rectifier 204. Closure of the transmit driver switch 210 gives rise to an abrupt increase in current in the implant coil 52 and AC-DC rectifier 204 of the implant power and communication subsystem 50, which, in turn, gives rise to a change in the induced voltage and current flow in external coil 90, and external power and communication subsystem 58. The voltage drop across the sense resistor 92 provides a means for monitoring the current flow in external power and communication subsystem 58 and external device 14 and thus provides a means for external microcontroller 56, onboard the external device 14, to decode the changes in current into digital data. The maximum rate of data transfer (i.e., baud rate) may be limited by the carrier frequency. In one example, at a carrier frequency of 100 kHz, a reasonable rate is 1200 baud.

Preferably, implant coil 52, FIGS. 3, 10, and 22, and external coil 90, FIGS. 11 and 22, are preferably placed in close proximity to each other, e.g., as shown in FIG. 22 and in further detail in FIG. 23 to provide sufficient inductive coupling between implant coil 52 and external coil 90 such that external power and communication subsystem 58 can wirelessly provide power to implant power and communication subsystem 50 and data can be wirelessly communicated to and from external power and communication subsystem 58 and implant power and communication subsystem 50, as discussed above.

In one embodiment, external coil 90 of external device 14 may be located relative to implant coil 52 of encapsulated implant 12 in human body 15, FIG. 1, using data wirelessly sent from implant power and communication subsystem 50 to external power and communication subsystem 58. For example, data communicated from implant power and communication subsystem 50 to external power and communication subsystem 58 includes the magnitude of the induced voltage (after rectification and filtering) onboard implant power and communication subsystem 50 of encapsulated implant 12, which provides a means by which a user of system 10 can position external device 14 and external coil 90, e.g., as shown in FIG. 1, relative to implant coil 52 of encapsulated implant 12 in human body 15. Preferably, the induced voltage onboard implant power and communication subsystem 50 is sufficient to both enable wireless communication and power transfer and to power the implant power and communication subsystem 50. Thus, the value of the induced voltage of implant coil 52 of implant power and communication subsystem 50 can be the basis for an intuitive, graphical display by external device 14 (discussed below) that enables the user to readily find an acceptable location for the external device 14 and to verify that sufficient coupling between implant coil 52 of encapsulated implant 12 and external coil 90 of external device 14 has been achieved for a calibration or flow measurement. Proper placement and orientation of external coil 90 of external device 14 and implant coil 52 of encapsulated implant 12 over the course of the flow rate measurement can be maintained by, inter alia, positioning and securing the external device 14 with apparel or by hand, such that the external coil 90 is positioned over implant coil 52, affixing the external coil 90 temporarily to the skin directly over the implant coil 52, e.g., using medical grade tape or adhesive, or longer term affixation, e.g., suturing, adhesive, of the external device 14 and external coil 90 to the skin over the encapsulated implant 12 for a period over which regular flow measurements will be needed.

Preferably, implant microcontroller 35, FIGS. 3 and 10, is configured to store the measured flow rate, the stored set of previously obtained calibration measurements, e.g. as shown in FIGS. 5 and 8, and identification information associated with the encapsulated implant 12, e.g., the serial number, model number, and the like, in a non-volatile manner.

In one example, external device 14, FIG. 22, includes display 290 which may display the measured flow, the previously obtained calibration measurements, the value of induced voltage on implant coil 52 or similar type measurements or values, and the identification information associated with encapsulated implant 12.

External device 14, FIGS. 1, 11, and 20, may include interface port 140 coupled to external microcontroller 56 configured to connect to computer subsystem 62, FIG. 11, by electrical cable 63. In another example, interface port 140 coupled to external microcontroller 56 may be configured to wirelessly connect computer subsystem 62. Interface port 140 coupled to external microcontroller 56 may also be configured to wirelessly connect to computer subsystem 62 configured as a smart device.

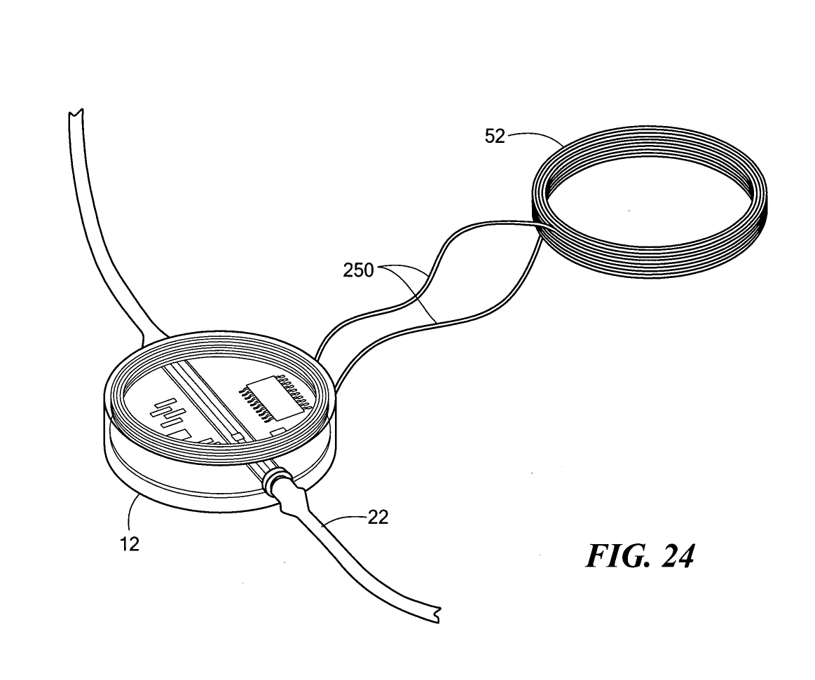

Although, as discussed thus far, implant coil 52, shown in one or more of FIGS. 2, 3, 22 and 23, is shown integrated with encapsulated implant 12, this is not a necessary limitation of this invention. In other embodiments, implant coil 52, FIG. 24, where like parts have been given like numbers, may be located remotely from encapsulated implant 12 as shown and coupled to encapsulated implant 12 with wires 250.

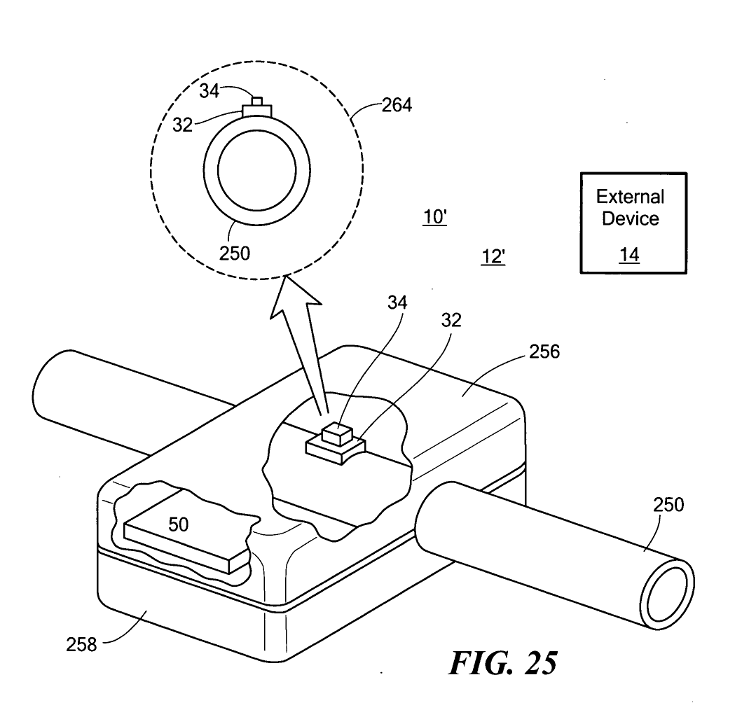

Although, as discussed above with reference to one or more of FIGS. 1-24 encapsulated implant 12 is shown having flow tube 22, FIG. 3, with inlet 24 and outlet 26 which receive flow of bodily fluid 28 in-line and heating element 32 and temperature sensor 34 and implant power and communication subsystem 50 are integrated in part of encapsulated implant 12, this is not a necessary limitation of this invention. In another embodiment, flow rate sensor system 10′, FIG. 25, where like parts have been given like numbers, for non-invasively measuring the flow rate of a bodily fluid includes external device 14 with external power and communication subsystem 58, having the same design as discussed above with reference to at least FIGS. 1, 11, and 20. However, in this embodiment, system 10′, FIG. 25, includes encapsulated implant 12′ that is clamped over a shunt, tube, vessel or catheter 250 implanted in a human body or animal body. Encapsulated implant 12′ clamps over shunt, tube, vessel or catheter 250 using clamshell device 252 with clamping members 256 and 258 as shown. Encapsulated implant 12′ includes heating element 32 and temperature sensor 34 and implant power and communication subsystem 50 having a similar structure as discussed above with reference to one or more of FIGS. 1-24. In this design, heating element 32, FIG. 25, externally and directly couples to shunt, tube, vessel or catheter 250, and temperature sensor 34 is directly externally coupled to heating element 32, e.g., as shown in blow-out caption 264. System 10′ operates similar to system 10, discussed above, with reference to one or more of FIGS. 1-24.

External device 14, FIGS. 1, 22, and 25, may be a dedicated unit, designed for measuring the flow rate of a bodily fluid, or may be a smart device, such as a phone or tablet with an App and attached coil accessory similar to external coil 90 and wires connecting it to external device 14.

The result is flow rate sensor system 10 and the method thereof, shown in one or more of FIGS. 1-25, that accurately and non-invasively measures the flow rate of a bodily fluid and provides a means of obtaining quantitative information on how a shunt, such as a VP shunt, or other similar type shunt, tube, vessel, or catheter, is functioning when implanted in a human or animal body. System 10 in some examples eliminate the need for obtaining cranial imaging using ultrasound, CT scanning, MRI, X-ray, and the like. Flow rate sensor system 10 can display or report the rate of flow of bodily fluids, such as CSF and other bodily fluids, and can be queried transcutaneously to allow the clinician to non-invasively assess shunt function during emergency room visits or during routine office visits. System 10 enables the primary care physician or specialist to see changes in the flow of bodily fluids over time and anticipate shunt failures prior to the development of symptoms. Thus, flow rate sensor system 10 and the method thereof enables timely intervention to maintain shunt function and reduce the likelihood of emergency shunt revision surgeries. Flow rate sensor system 10 and the method thereof can measure and monitor flow rate of bodily fluids in a patient with a shunt who arrives at the emergency room with symptoms possibly indicative of shunt failure and quickly and accurately provide the clinician with information regarding shunt function and, thus, can avoid unnecessary diagnostic or surgical procedures. The result is better care, reduced risk of death or injury from shunt failure, and reduced cost of care for those whose lives depend on continuous and proper function of their shunts.

External device 14 enables the clinician to obtain and store a “snapshot” of flow rate of CSF or other bodily fluids whenever needed. Because patient posture and orientation can affect flow through a shunt, the clinician can choose to place patient in various orientations and then take a flow rate measurement at selected orientations. The external device or external coil can be affixed to the patient to enable automatically-initiated, periodic measurements and storage of the flow rates of bodily fluids, such as CSF, over an extended time period. This allows the clinician to see any trends in the flow characteristics of the shunt over a desired period of time. For example, CSF flow rate measurements could be automatically obtained every half hour to monitor shunt function, both in the hospital and after discharge, for the critical days following a shunt placement or a shunt revision surgery. In a second example, a CSF flow rate measurement could be taken every 5 minutes on a shunted patient who arrives at the emergency room with symptoms possibly indicative of shunt failure. This would give the clinician complete knowledge of the flow characteristics of the shunt, possibly preventing unnecessary diagnostic or surgical procedures, including MRI or CT imaging and shunt revisions.

For enablement purposes only, the computer program listing appendix provided can be executed on implant microcontroller 35 and external microcontroller 56 to carry out the primary steps and/or functions of flow rate sensor system 10 shown in one or more of FIGS. 1-25 and recited in the claims hereof. Other equivalent algorithms and code can be designed by a software engineer and/or programmer skilled in the art, using the information provided herein.

Although specific features of the invention are shown in some drawings and not in others, this is for convenience only as each feature may be combined with any or all of the other features in accordance with the invention. The words “including”, “comprising”, “having”, and “with” as used herein are to be interpreted broadly and comprehensively and are not limited to any physical interconnection. Moreover, any embodiments disclosed in the subject application are not to be taken as the only possible embodiments. Other embodiments will occur to those skilled in the art and are within the following claims.

In addition, any amendment presented during the prosecution of the patent application for this patent is not a disclaimer of any claim element presented in the application as filed: those skilled in the art cannot reasonably be expected to draft a claim that would literally encompass all possible equivalents, many equivalents will be unforeseeable at the time of the amendment and are beyond a fair interpretation of what is to be surrendered (if anything), the rationale underlying the amendment may bear no more than a tangential relation to many equivalents, and/or there are many other reasons the applicant cannot be expected to describe certain insubstantial substitutes for any claim element amended.

Other embodiments will occur to those skilled in the art and are within the following claims.

What is claimed is:

1. A flow rate sensor system for non-invasively measuring the flow rate of a bodily fluid, the system comprising:
an encapsulated implant including:
a flow tube having an inlet and an outlet configured to receive a flow of a bodily fluid,
a heating element externally coupled to the flow tube configured to dissipate heat at a predetermined rate over a predetermined amount of time,
a temperature sensor externally coupled to the heating element configured to measure a temperature rise of the heating element over the predetermined amount of time,
an implant microcontroller coupled to the temperature sensor configured to determine the flow rate of the bodily fluid in the flow tube from the measured temperature rise of the heating element over the predetermined amount of time and a curve fit to a stored set of previously obtained calibration measurements, and
an implant power and communication subsystem coupled to the implant microcontroller configured to wirelessly receive power and wirelessly transmit and receive data; and
an external device including:
an external microcontroller, and
an external power and communication subsystem coupled to the external microcontroller configured to wirelessly deliver power to the implant power and communication subsystem and transmit and receive data to and from the implant power and communication subsystem.

2. The system of claim 1 in which the temperature sensor includes a thermistor.

3. The system of claim 1 in which the temperature sensor includes a resistance temperature detector (RTD).

4. The system of claim 1 further including a thermistor configured as both the temperature sensor and the heating element.

5. The system of claim 1 in which the temperature sensor includes a thermocouple.

6. The system of claim 1 in which the heating element includes a surface mount resistor.

7. The system of claim 1 in which the heating element includes a coil of electrically conductive wire.

8. The system of claim 1 in which the heating element includes a printed circuit heater.

9. The system of claim 1 in which the heating element is directly attached to the external surface of the flow tube.

10. The system of claim 1 further including a thermal insulator configured to thermally isolate the heating element and the temperature sensor from cooling paths other than the direct cooling path to the bodily fluid in the flow tube.

11. The system of claim 10 in which the thermal insulator includes an insulation layer over the heating element and the temperature sensor.

12. The system of claim 10 in which the thermal insulator includes a sealed volume of air surrounding the heating element and the temperature sensor.

13. The system of claim 1 in which the flow through flow tube is comprised of a thin wall of polymer material with low thermal conductivity configured to limit heat transfer along a length and a circumference of the tube while maintaining heat transfer in a radial direction to the fluid.

14. The system of claim 1 in which the bodily fluid includes one or more of cerebrospinal fluid (CSF), bile, blood, and urine.

15. The system of claim 1 in which the encapsulated implant is coupled to a shunt, tube, vessel or catheter implanted in a human body or an animal.

16. The system of claim 15 in which the shunt includes one or more of: a ventriculo-peritoneal (VP) shunt, ventroarterial shunt, and lumboperitoneal shunt.

17. The system of claim 16 in which the encapsulated implant is coupled to a distal catheter of the shunt.

18. The system of claim 16 in which the encapsulated implant is coupled to a proximal catheter of the shunt.

19. The system of claim 1 in which the heating element and the temperature sensor are located proximate the outlet.

20. The system of claim 1 in which the heating element and the temperature sensor are located proximate the inlet.

21. The system of claim 1 in which the heating element and the temperature sensor are located between the inlet and the outlet.

22. The system of claim 1 in which the external power and communication subsystem includes an external coil coupled to the external microcontroller and the implant power and communication subsystem includes an implant coil coupled to the microcontroller.

23. The system of claim 22 in which the implant coil of the encapsulated implant is located using magnitude of the induced voltage wirelessly sent from the implant coil to the external coil.

24. The system of claim 22 in which the external coil is positioned proximate and in alignment with the implant coil to achieve sufficient inductive coupling between the external coil and the implant coil.

25. The system of claim 22 in which the external coil is remotely located from and tethered to the external power and communication subsystem.

26. The system of claim 22 in which the implant coil is integrated with the encapsulated implant.

27. The system of claim 22 in which the implant coil is remotely located from and tethered to the encapsulated implant.

28. The system of claim 24 in which the external power and communication subsystem includes a resonant circuit comprised of the external coil and a capacitor, and a source of low-level voltage pulses, the external device resonant circuit configured to provide sinusoidal current in the external coil of sufficient amplitude to induce sufficient sinusoidal voltage in the implant coil.

29. The system of claim 28 in which the implant power and communication subsystem includes an implant resonant circuit comprised of the implant coil and a capacitor having a resonance frequency closely matched to the resonance frequency of the external resonant circuit to maintain sufficient AC voltage amplitude to power the implant power and communication subsystem and to enable communication between the external power and communication subsystem and implant power and communication subsystem.

30. The system of claim 29 in which the implant power and communication subsystem is configured to convert induced sinusoidal voltages in the implant coil to a highly regulated DC voltage over the range of loading conditions to power the heating element, the temperature sensor, the microcontroller, and components of the implant power and communication subsystem.

31. The system of claim 29 in which the external power communication subsystem is configured to enable the external microcontroller to communicate data to the implant power and communication subsystem by changing the voltage supplied to the resonant circuit of the external power and communication subsystem to modulate the amplitude of the voltage induced in the implant coil and use that change in voltage to represent different binary states.

32. The system of claim 31 in which the implant power and communication subsystem transmits binary values serially to the external power and communication subsystem by sequentially applying and removing an electrical load from the implant coil to induce changes in voltage in the external coil that are decoded into data by the external microcontroller.

33. The system of claim 32 in which the external power and communication subsystem includes a sense resistor configured to measure change in the amplitude of the current in external power and communication subsystem resulting from changes in the induced voltage in the external coil.

34. The system of claim 33 in which the external microcontroller is coupled to the series resistor and is configured to decode changes in the current of the external power and communication subsystem into data.

35. The system of claim 1 in which the implant microcontroller is configured to store the set of previously obtained calibration measurements relating heating element temperature rise to flow rate.

36. The system of claim 1 in which the implant microcontroller is configured to determine when the temperature of the heating element is no longer rising to minimize the length of time needed to determine the flow rate.

37. The system of claim 1 in which the implant microcontroller is configured to determine the flow rate from the measured temperature rise when temperature of the heating element is determined to be no longer rising to minimize the length of time needed to determine the flow rate, the amount of heat generated by the heating device, and the amount of heat delivered to a patient.

38. The system of claim 1 in which the implant microcontroller is configured to store identification information associated with the encapsulated implant.

39. The system of claim 1 in which the external device includes an interface port coupled to the external microcontroller configured to connect to a computer subsystem by an electrical cable.

40. The system of claim 1 in which the external device includes an interface port coupled to the external microcontroller configured to wirelessly connect to a computer subsystem.

41. The system of claim 1 in which the external device includes an interface port coupled to the external microcontroller configured to wirelessly connect to a smart device.

42. The system of claim 1 in which the implant microcontroller is configured to use a mean value of a set of temperature rise samples obtained over the predetermined amount of time as the temperature rises to determine the flow rate of the bodily fluid in order to increase the signal to noise ratio.

43. The system of claim 1 in which the implant microcontroller is configured to use a weighted average of a set of temperature rise samples obtained over the predetermined amount of time as the temperature rises to determine the flow rate of the bodily fluid in order to increase the signal to noise ratio.

44. The system of claim 1 in which the encapsulated implant is implanted in a human body.

45. The system of claim 1 in which the external device includes a smart device including a flow sensor App and a tethered external coil.

46. The system of claim 1 in which the external device includes a display for displaying one or more of: the measured flow rate, the predetermined amount of time, induced voltage on the implant coil, and identification information associated with the encapsulated implant.

47. A flow rate sensor system for non-invasively measuring the flow rate of a bodily fluid, the system comprising:
an encapsulated implant including:
a flow tube having an inlet and an outlet configured to receive a flow of a bodily fluid,
a heating element externally coupled to the flow tube configured to dissipate heat at a predetermined rate over a predetermined temperature rise of the heating element,
a temperature sensor externally coupled to the heating element configured to measure a temperature drop of the heating element over a predetermined amount of time of cooling,
an implant microcontroller coupled to the temperature sensor configured to determine the flow rate of the bodily fluid in the flow tube from the measured temperature drop of the heating element over the predetermined amount of cooling time and a curve fit to a stored set of previously obtained calibration measurements, and
an implant power and communication subsystem coupled to the implant microcontroller configured to wirelessly receive power and wirelessly transmit and receive data; and
an external device including:
an external microcontroller, and
an external power and communication subsystem coupled to the external microcontroller configured to wirelessly deliver power to the implant power and communication subsystem and transmit and receive data to and from the implant power and communication subsystem.

48. A flow rate sensor system for non-invasively measuring the flow rate of a bodily fluid, the system comprising:
an encapsulated implant including:
a heating element externally coupled to a shunt, catheter, tube, or vessel configured to receive a flow of a bodily fluid, the heating element configured to dissipate heat at a predetermined rate over a predetermined amount of time;
a temperature sensor externally coupled to the heating element configured to measure a temperature rise of the heating element over the predetermined amount of time,
an implant microcontroller coupled to the temperature sensor configured to determine the flow rate of the bodily fluid in the shunt, catheter, tube or vessel from the measured temperature rise of the heating element over the predetermined amount of time and a curve fit to a stored set of previously obtained calibration measurements; and
an implant power and communication subsystem coupled to the implant microcontroller configured to wirelessly receive power and wirelessly transmit and receive data; and
an external device including:
an external microcontroller, and
an external power and communication subsystem coupled to the external microcontroller configured to wirelessly deliver power to the implant power and communication subsystem and transmit and receive data to and from the implant power and communication subsystem.

49. The system of claim 48 in which the encapsulated implant is configured as a two-piece clamp externally coupled to the shunt, catheter, tube, or vessel.

50. A flow rate sensor system for non-invasively measuring the flow rate of a bodily fluid, the system comprising:
an encapsulated implant including:
a heating element externally coupled to the shunt, catheter, tube, or vessel configured to receive a flow of a bodily fluid the heating element configured to dissipate heat at a predetermined rate over a predetermined temperature rise of heating element;
a temperature sensor externally coupled to the heating element configured to measure a temperature drop of the heating element over a predetermined amount of time of cooling,
an implant microcontroller coupled to the temperature sensor configured to determine the flow rate of the bodily fluid in the shunt, catheter, tube or vessel from the measured temperature drop of the heating element over the predetermined amount of cooling time and a curve fit to a stored set of previously obtained calibration measurements, and
an implant power and communication subsystem coupled to the implant microcontroller configured to wirelessly receive power and wirelessly transmit and receive data; and
an external device including:
an external microcontroller, and
an external power and communication subsystem coupled to the external microcontroller configured to wirelessly deliver power to the implant power and communication subsystem and transmit and receive data to and from the implant power and communication subsystem.

51. The system of claim 50 in which the encapsulated implant is configured as a two-piece clamp externally coupled to the shunt, catheter, tube, or vessel.

52. A method for non-invasively measuring the flow rate of a bodily fluid, the method comprising:
providing an encapsulated implant coupled to a shunt, catheter, tube or vessel;
receiving a flow of a bodily fluid in the shunt, catheter, tube or vessel;
externally coupling a heating element to the shunt, catheter, tube or vessel configured to dissipate heat at a predetermined rate over a predetermined amount of time;
externally coupling a temperature sensor to the heating element;
measuring a temperature rise of the heating element over a predetermined amount of time;
determining the flow rate of the bodily fluid in the shunt, catheter, tube or vessel from the measured temperature rise and a curve fit to a stored set of previously obtained calibration measurements;
providing an external device;
wirelessly delivering power from the external device to the encapsulated implant; and
wirelessly transmitting and receiving data to and from the encapsulated implant and the external device.

53. The method of claim 52 further including thermally isolating the heating element and the temperature sensor.

54. The method of claim 52 further including locating the encapsulated implant, using data wirelessly sent from the encapsulated implant to the external device.

55. The method of claim 52 further including positioning an external coil of the external device proximate and in alignment with an implant coil of the encapsulated implant to provide sufficient inductive coupling between an external coil of the external device and an implant coil.

56. The method of claim 52 further including storing on a microcontroller of the encapsulated implant the set of previously obtained calibration measurements.

57. The method of claim 52 further including storing on a microcontroller of the encapsulated implant identification information associated with the encapsulated implant.

58. The method of claim 52 further including determining the flow rate from a current measured temperature rise when the temperature of the heating element is determined to be no longer rising to minimize the length of time needed to determine the flow rate, the amount of heat generated by the heating device, and the amount of heat delivered to a patient.

59. A method for non-invasively measuring the flow rate of a bodily fluid, the method comprising:
providing an encapsulated implant coupled to a shunt, catheter, tube or vessel;
receiving a flow of a bodily fluid in the a shunt, catheter, tube or vessel;
externally coupling a heating element to the shunt, catheter, tube or vessel configured to dissipate heat until a predetermined temperature rise is achieved;
externally coupling a temperature sensor to the heating element;
measuring a temperature drop of the heating element over a predetermined amount of time of cooling;
determining the flow rate of the bodily fluid in the flow tube from the measured temperature drop and a curve fit to a set of previously obtained calibration measurements;
providing an external device;
wirelessly delivering power to the encapsulated implant; and
wirelessly transmitting and receiving data to and from the encapsulated implant.
